(19) United States
(12) Patent Application Publication
(10) Pub. No.: US 2007/0020315 A1
(43) Pub. Date: Jan. 25, 2007

(54) Tissue products having low stiffness and antimicrobial activity

(75) Inventors: Thomas Gerard Shannon, Neenah, WI (US); David Andrew Moline, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/189,483

(22) Filed: Jul. 25, 2005

(51) Int. Cl. A61K 9/70; A61K 31/19; A61K 31/192

(52) U.S. Cl. 424/443; 514/557; 514/568; 514/574

(57) ABSTRACT

The inner plies of a multi-ply antimicrobial tissue product are selectively weakened by chemical or mechanical means to improve the overall softness of the tissue product. The addition of a deliquescent salt to the inner ply or plies containing the antimicrobial agent is particularly advantageous.

### BACKGROUND OF THE INVENTION

Antimicrobial tissue products, such as virucidal facial tissue, have been produced in a three-ply product form in which the inner ply is treated with an aqueous solution of an antimicrobial agent. While effective, the application of the antimicrobial agent increases the stiffness of the center ply due to the creation of hydrogen bonding when the applied antimicrobial agent dries. This in turn increases the stiffness of the product and detracts from its overall softness.

Since one component of softness is the surface feel of the product, one approach to improve the softness of such a product is to provide an outer surface of the product with an irritation-inhibiting composition. While incorporation of a topical softener to the outer plies of the antimicrobial tissue product may increase the surface softness of the product, it does not completely compensate for the negative impact on the softness of the tissue product due to the presence of the antimicrobial compound.

Thus, there is a need to further improve the softness of such products, particularly with regard to reducing the stiffness of the center ply created by the presence of the antimicrobial agent and particularly in a manner that does not compromise the efficacy and cost of the product.

### SUMMARY OF THE INVENTION

It has now been discovered that the softness of anti-microbial tissues can be improved by incorporating an additional chemical agent into the inner ply or plies that contain the antimicrobial agent without compromising the antimicrobial efficacy and/or by mechanically weakening the inner ply or plies containing the antimicrobial agent.

Hence in one aspect, the invention resides in a multi-ply tissue product comprising two outer plies and one or more inner plies containing an antimicrobial agent, wherein said one or more inner plies containing an antimicrobial agent have a geometric mean tensile strength which is less than the geometric mean tensile strength of the outer plies. The lower tensile strength can be achieved by mechanical or chemical treatments.

In another aspect, the invention resides in a method of making a multi-ply antimicrobial tissue product comprising: (a) providing two outer tissue plies and an inner tissue ply; (b) adding an antimicrobial agent to the inner ply; (c) chemically or mechanically weakening the inner ply; and (d) combining the two outer plies and the inner ply to form a multi-ply tissue product.

Specifically, the geometric mean tensile strength of the ply or plies containing the antimicrobial agent can be from about 10 to about 75 percent less than the tensile strength of the outer plies of the tissue product, more specifically from about 10 to about 50 percent less, and still more specifically from about 15 to about 30 percent less.

Particularly suitable means for selectively reducing the tensile strength and/or stiffness of the inner ply or plies containing the antimicrobial agent include, either alone or in combination with each other, the addition of a deliquescent salt, the use of a debonder applied in the wet end of the tissue making process, calendering after the application of the antimicrobial agent, or by aperturing the ply such that the apertures create regions of weakness. Various means for aperturing the plies are well known to those skilled in the tissue making art, which include perf embossing the web with a pin embossing pattern or otherwise creating a high level of shear in the web by the design of the male and female embossing elements.

### DETAILED DESCRIPTION OF THE INVENTION

As used herein, a “tissue product” is any product suitable as a facial tissue, bath tissue, paper towel, table napkin and the like.

As used herein, the term “antimicrobial agent” includes any of the virucides, bacteriocides, germicides, fungicides and disinfectants known in the art. The selection of any particular antimicrobial agent will be dependent on its efficacy versus relevant microorganisms, human safety and toxicological profile, and environmental safety and toxicological profile. Suitable virucidal compositions include, without limitation, the carboxylic acid or the carboxylic acid/surfactant compositions disclosed in U.S. Pat. No. 4,975,217, issued to Brown-Skrobot et al.; U.S. Pat. No. 4,828,912, issued to Hossain et al.; U.S. Pat. No. 4,897,304, issued to Hossain et al.; U.S. Pat. No. 4,764,418, issued to Kuenn et al.; and U.S. Pat. No. 4,738,847, issued to Rothe et al., all of which are herein incorporated by reference.

Particularly suitable antimicrobial agents include carboxylic acids having the structure:
R—COOH
wherein R is a radical selected from the group consisting of C₁-C₆ alkyl, substituted C₁-C₆ alkyl, carboxy C₁-C₆ alkyl, carboxyhydroxy C₁-C₆ alkyl, carboxy halo C₁-C₆ alkyl, carboxy dihydroxy C₁-C₆ alkyl, dicarboxyhydroxy C₁-C₆ alkyl, C₁-C₆ alkenyl, carboxy C₁-C₆ alkenyl, dicarboxy C₁-C₆ alkenyl, phenyl, and substituted phenyl radicals. The hydrogen atoms of any of the above compounds may be substituted by one or more functional groups such as halogen atoms, hydroxyl groups, amino groups, thiol groups, nitro groups, cyano groups, and the like.

Other suitable antimicrobial agents include, without limitation, compounds having the structure:
R—COOR′
wherein “R” is selected from the group consisting of: a radical selected from the group consisting of C₁-C₆ alkyl, substituted C₁-C₆ alkyl, carboxy C₁-C₆ alkyl, carboxyhydroxy C₁-C₆ alkyl, carboxy halo C₁-C₆ alkyl, carboxy dihydroxy C₁-C₆ alkyl, dicarboxyhydroxy C₁-C₆ alkyl, C₁-C₆ alkenyl, carboxy C₁-C₆ alkenyl, dicarboxy C₁-C₆ alkenyl, phenyl, and substituted phenyl radicals; and
“R′” is selected from the group consisting of: hydrogen; halogen; hydroxyl groups; amino groups; thiol groups; nitro groups; and cyano groups.

More specifically, particularly suitable antimicrobial agents include the following organic acids: citric acid; malic acid; malic acid; tartaric acid; salicylic acid; glycolic acid; adipic acid; glutaric acid; succinic acid; benzoic acid; lactic-acid; and mixtures thereof. Alphahydroxy and betahydroxy acids are also suitable.

The antimicrobial agent, particularly carboxylic acids, can be combined with a surfactant. Carboxylic acid/surfactant antimicrobial agents are effective at add-on rates as low as 0.5 milligrams per square inch of tissue. The surfactant can be cationic, anionic, or nonionic. The nonionic surfactants can include, without limitation, the polyoxyethylenated alkylphenols, such as TRITON X-100® manufactured by Union Carbide of Danbury, Conn., and the polyoxyethylenated sorbitol esters, such as TWEEN 40®), manufactured by Uniquema of Wilmington, Del. The cationic surfactants can include, without limitation, cetylpyridinium chloride (C₅H₅N⁺(CH₂)₁₅ CH₃Cl⁻), dimethylbenzethonium quaternary ammonium chloride (Me₃CCH₂C(Me)₂C₆H₃(Me)—OCH₂ CH₂ OCH₂CH₂⁺N(Me)₂H₂C₆H₅Cl⁻). The anionic surfactants can be represented by the structures:
(ROSO₃)ₓ M⁺ or (RSO₃)ₓ M⁺
wherein, M⁺ is a mono-, di- or tri-valent metal cation or an ammonium or substituted ammonium ion; x is an integer; and R is an alkyl group; or 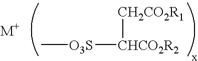
wherein, M⁺ and x are defined as above and R₁ and R₂ may be the same or different and may be represented by straight or branched chain aliphatic groups.

More specifically, the anionic surfactants include secondary alkane sulfonates and sarcosinate surfactants. In some embodiments of the present invention, the anionic surfactants may include sodium dodecyl sulfate (CH₃ (CH₂)₁₀—CH₂ OSO₃—Na), and the 1,4-bis (2-ethylhexyl) ester, sodium salt of sulfosuccinic acid, as manufactured by Cytec Industries of West Paterson, N.J., under the tradename of AEROSOL OT. The above surfactants are presented in an illustrative rather than a limiting sense.

The antimicrobial agent can be applied to the tissue ply in any suitable manner, such as by wet-end addition, embossing, spraying, coating, dipping, printing, or the like. The application of the antimicrobial agent may be uniform, in discreet modified zones, or other patterns such as stripes, dots, corrugated patterns, and the like.

In order to further optimize the antimicrobial effectiveness of the tissue product, blends of two or more of the antimicrobial agents can be applied to the inner tissue ply or plies. In one particular example, a blend of citric acid and malic acid may be used. The ratio of the citric acid to the malic acid can be from about 10 to about 1, more specifically about 1 to 1 or, alternatively, from about 1 to about 10.

The antimicrobial agent can be present in the tissue product in any amount which is antimicrobially effective. The term “antimicrobially effective amount” means an amount sufficient to cause a 3 log drop in rhinovirus type 16 within 20 minutes in accordance with the Virucidal Assay Test described in the above-identified U.S. Pat. No. 4,897,304. Specifically, the add-on amount of the antimicrobial agent in the tissue ply can be from about 0.1 to about 10 milligrams per square inch (mg/in²), more specifically from about 0.3 to about 8.0 mg/in² and still more specifically from about 0.5 to about 5.0 mg/in². Stated differently, the add-on amount of the antimicrobial agent in a given tissue ply can be from about 0.5 to about 15 weight percent based on dry fiber, more specifically from about 3 to about 12 weight percent, and still more specifically from about 5 to about 10 weight percent.

As used herein, a “deliquescent salt” is any solid material that can absorb a sufficient amount of moisture from the air to form a solution or any liquid material that can absorb greater than 50% by weight of water from the air to form a homogeneous aqueous solution. While any deliquescent salt can be used for purposes of this invention, suitable deliquescent salts include certain inorganic salts, such as calcium chloride, lithium chloride, lithium bromide, sodium acetate, potassium acetate and ammonium acetate, and certain organic salts, such as trimethylamine n-oxide.

The amount of the deliquescent salt in an inner ply containing an antimicrobial agent can be any amount that provides the desired equilibrium moisture content. More specifically, the amount can be from about 2 to about 150 percent by weight of dry fiber or greater, more specifically from about 2 to about 125 dry weight percent, more specifically from about 3 to about 125 dry weight percent, more specifically from about 5 to about 100 dry weight percent, more specifically from about 5 to about 75 dry weight percent, more specifically from about 5 to about 50 dry weight percent and still more specifically from about 10 to about 50 dry weight percent. The specific add-on amount of the deliquescent salt serves to deliver the desired equilibrium moisture content and will depend upon the specific deliquescent salt selected.

As used herein, the “equilibrium moisture content” represents the moisture content of a tissue sheet at 50% relative humidity and 25° C. (standard TAPPI conditions). At equilibrium, the amount of moisture within the sheet will not change with time at the same humidity and temperature condition. The equilibrium moisture content is expressed as a weight percent of the dry sheet including the deliquescent salt and any additional non-volatile components. The equilibrium moisture content in the sheet can be controlled by the absorbent capacity of the sheet, the amount of water on a percent basis that the deliquescent salt absorbs and the amount of deliquescent salt in the sheet. The equilibrium moisture content of the ply comprising the antimicrobial agent and the deliquescent salt can be from 8 percent to about 50 dry weight percent, more specifically from about 10 to about 40 dry weight percent and still more specifically from about 10 to about 30 dry weight percent. In a specific embodiment, the deliquescent salt is applied only to the ply or plies containing the antimicrobial agent such that the ply or plies comprising the antimicrobial agent has an equilibrium moisture content that can be from about 30 to about 1000 percent or greater than the equilibrium moisture content of the ply or plies not containing the antimicrobial agent, more specifically from about 80 to about 500 percent greater than the equilibrium moisture content of the ply or plies not containing the antimicrobial agent. The equilibrium moisture content of the plies not containing the antimicrobial agent will range from about 0.5 percent by weight of dry fibers to about 8 percent by weight of dry fibers, more specifically from about 1 to about 7 percent, and still more specifically from about 1.5 to about 6 percent.

The deliquescent salt can be incorporated into the targeted tissue ply by any suitable means, such as spraying or, if the sheet is made by a wet-laying process, incorporating the deliquescent salt into the water used to suspend the fibers prior to sheet formation. Additionally, the deliquescent salt can be added to the sheet as a neat liquid or a solid. The deliquescent salt will then absorb moisture from the air and distribute throughout the sheet.

In selecting the appropriate deliquescent salt, the selection should be such that no undesirable chemical reaction occurs between the deliquescent salt and the antimicrobial agent. In particular, in the case of an antimicrobial agent containing a carboxylic acid and a surfactant, care should be taken to avoid formation of insoluble precipitates between the surfactant and the deliquescent salt. For example, calcium chloride will react with sodium lauryl sulfate to form sodium chloride and an insoluble precipitate of calcium lauryl sulfate. In doing so, the efficaciousness of the surfactant and the deliquescent properties of the salt are destroyed.

In one embodiment, the deliquescent salt is selected from deliquescent salts of the group IA metals. Examples of such deliquescent salts include lithium bromide, lithium chloride, potassium acetate, and mixtures thereof. Salts of the group IA metals are particularly preferred in their lack of ability to form insoluble precipitates with many of the surfactants that are found to be most efficacious, such as sodium lauryl sulfate. At other times it may be advantageous for the surfactant and the deliquescent salt to comprise the same cationic species. By comprising the same cationic species, ion transfer reactions that may negatively impact the deliquescent or solubility behavior of the individual species can be eliminated. In general, however, it is found that it is satisfactory to have the cationic species be of the same periodic table group. In general, anionic surfactants tend to comprise cationic species of the group I elements. Group II cations tend to form insoluble materials with the anionic species typically associated with anionic surfactants. Thus, if a deliquescent salt of a group IIA metal ion such as calcium chloride is used, the surfactant should be selected such that the surfactant is preferably non-ionic or cationic. For example, if an anionic surfactant is selected along with a deliquescent salt comprising a group IIA cation, the surfactant should be selected such that the group IIA salt of the surfactant is water soluble and preferably that the cationic species of the anionic surfactant be comprised of a group IIA metal.

For example, in one embodiment it may be preferred to use an anionic surfactant selected from the group of secondary alkane sulfonates and sarcosinate surfactants in combination with the carboxylic acid. Such anionic surfactants in this group include, but are not limited to, sodium lauryl sulfate, sodium dodecyl sulfate (CH₃ (CH₂)₁₀—CH₂ OSO₃—Na), and the 1,4-bis (2-ethylhexyl) ester, sodium salt of sulfosuccinic acid, as manufactured by Cytec Industries of West Paterson, N.J., under the tradename of AEROSOL OT. Such anionic surfactants will form insoluble salts with group IIA metal ions such as calcium and magnesium. In this case it is preferred that a deliquescent salt such as lithium chloride, lithium bromide, potassium acetate or combinations thereof be used.

When calcium and magnesium deliquescent salts, such as calcium chloride and magnesium chloride, are used it is preferable to use such non-ionic surfactants as the polyoxyethylenated alkylphenols, such as TRITON X-100® manufactured by Union Carbide of Danbury, Connecticut, and the polyoxyethylenated sorbitol esters, such as TWEEN 40®, manufactured by Uniquema of Wilmington, Delaware or a cationic surfactants such as but not limited to cetylpyridinium chloride (C₅H₅N⁺(CH₂)₁₅ CH₃Cl⁻), dimethylbenzethonium quaternary ammonium chloride (Me₃CCH₂C(Me)₂C₆H₃(Me)—OCH₂ CH₂ OCH₂CH₂⁺N(Me)₂H₂C₆H₅Cl⁻) and mixtures thereof. When cationic surfactants are employed it may be advantageous that the anionic species of the cationic surfactant and the anionic species of the deliquescent salt be the same. For example, cetylpyridinium chloride could be advantageously combined with the deliquescent salt calcium chloride.

As used herein, a “debonder” refers to a chemical species that softens or weakens a tissue sheet by preventing the formation of hydrogen bonds during the drying of the sheet. Examples of such debonders and softening chemistries are broadly taught in the art. Exemplary compounds include the simple quaternary ammonium salts having the general formula (R^{1′})_{4-b}—N⁺—(R^{1″})_{b} X⁻ wherein R1′ is a C₁₋₆ alkyl group, R1″ is a C₁₄-C₂₂ alkyl group, b is an integer from 1 to 3 and X⁻ is any suitable counter ion. Other similar compounds include the monoester, diester, monoamide and diamide derivatives of the simple quaternary ammonium salts. A number of variations on these quaternary ammonium compounds are known and should be considered to fall within the scope of the present invention. Additional softening compositions include cationic oleyl imidazoline materials such as methyl-1-oleyl amidoethyl-2-oleyl imidazolinium methyl sulfate commercially available as Mackernium CD-183 from McIntyre Ltd., located in University Park, Ill. and Prosoft TQ-1003 available from Hercules, Inc. Such softeners may also incorporate a humectant or a plasticizer such as a low molecular weight polyethylene glycol (molecular weight of about 4,000 Daltons or less) or a polyhydroxy compound, such as glycerin or propylene glycol.

Preferably these debonders are applied to the fibers in the wet end of the tissue making process, that is, while the fibers are in an aqueous slurry prior to tissue sheet formation, to aid in bulk softness. The amount of debonder used in center ply or plies comprising the antimicrobial agent can be any amount that achieves the appropriate tensile strength. In general, the amount of debonder can range from about 0.05 to about 2 percent by weight of dry fibers in the ply or plies comprising the antimicrobial agent, more specifically from about 0.1 to about 1.5 percent, and still more specifically from about 0.2 to about 1 percent. The amount of debonder in the ply or plies comprising the antimicrobial agent is selected such that the tensile strength of the ply is from about 10 to about 75 percent less than that of the outer plies of the tissue sheet not comprising the antimicrobial agent, more specifically from about 15 to about 60 percent less, and still more specifically from about 20 to about 50 percent less. By maintaining the strength of the outer plies, the level of lint and slough produced by the tissue in use is less than if the debonder is applied to the outer plies of the tissue sheet. At the same time, the overall bulk softness of the tissue is improved.

The dry tensile strength of the products of the present invention can be any suitable level. In general, the tissue products of the present invention will have geometric mean tensile strengths ranging from about 500 to about 2000 grams per 3 inches, more specifically from about 600 to about 1700 grams per 3 inches, and still more specifically from about 700 to about 1300 grams per 3 inches. When a weaker center ply comprising the antimicrobial agent is used, the center ply of the tissue sheet may have a geometric mean tensile strength of from about 55 to about 620 grams per 3 inches, more specifically from about 85 to about 595 grams per 3 inches, and still more specifically from about 100 to about 570 grams per 3 inches. The tensile strength of the outer plies not comprising the antimicrobial agent suitably ranges from about 175 to about 890 grams per 3 inches, more specifically from about 180 to about 830 grams per 3 inches, and still more specifically from about 190 to about 800 grams per 3 inches. It should be appreciated that, when referring to the tensile strengths of the multi-ply product, the tensile strength is representative of the tensile strength as measured on the entire multi-ply product representing 2 or more plies. On the other hand, when referring to the tensile strengths of the individual plies, the tensile strength refers to the tensile strength as measured on an individual ply and not multiple plies.

In addition to adding debonders, the tensile strength of the ply comprising the antimicrobial agent may be reduced by selectively mechanically weakening the center ply. Such weakening may be done either prior to, during or after application of the antimicrobial agent. The particular method of creating the strength degradation is not overly critical to the invention so long as the above criteria for tensile difference between the plies are met. A variety of mechanical methods such as calendering, breaker bars and s-wrap are known in the art and are suitable for reducing the tensile strength of the ply comprising the antimicrobial agent.

### Test Methods

Geometric Mean Tensile Strength

The geometric mean tensile (GMT) strength is expressed as grams-force per 3 inches of sample width. GMT is computed from the peak load values of the MD (machine direction) and CD (cross-machine direction) tensile curves, which are obtained under laboratory conditions of 23.0° C.±1.0° C., 50.0±2.0% relative humidity after the tissue sheet has equilibrated to the testing conditions for a period of not less than 4 hours. Testing is conducted on a tensile testing machine maintaining a constant rate of elongation and the width of each specimen tested is 3 inches. The “jaw span” or the distance between the jaws, sometimes referred to as gauge length, is 4.0 inches (50.8 mm). The crosshead speed is 10 inches per minute (254 mm/min.) A load cell or full-scale load is chosen so that all peak load results fall between 10 and 90 percent of the full-scale load. A suitable system is an Instron 1122 tensile frame connected to a Sintech data acquisition and control system utilizing IMAP software running on a “486 Class” personal computer or equivalent. This data system records at least 20 load and elongation points per second. A total of 10 specimens per sample are tested with the sample mean being used as the reported tensile value. The geometric-mean tensile is calculated from the following equation:
GMT=(MD Tensile*CD Tensile)^{1/2}

Multi-ply products are tested as multi-ply products and results represent the tensile strength of the total product. For example, a 3-ply product is tested as a 3-ply product and recorded as such. Testing of individual plies is done by cutting the sample specimens and separating each of the plies. Each ply is then tested separately and the tensile strength of each individual ply recorded. MD and CD tensile strengths are recorded and the GMT calculated as above. A total of 10 specimens per sample are tested with the sample mean being used as the reported tensile value. At times, the strength of the ply comprising the antimicrobial agent may be insufficient for testing of a single ply. In such cases, two or more plies may be tested according to the above procedure. The tensile strength of a single ply is then determined by taking the tensile strength of the multiple plies and dividing by the number of plies.

Equilibrium Moisture Content

The equilibrium moisture content of the individual tissue plies is determined as follows. First, the plies are segregated into plies comprising the antimicrobial composition and plies not containing the antimicrobial composition. Samples of the segregated plies are placed in a 100° C. oven and air-dried for 1 hour. Sample sizes of 2-3 grams are selected, although larger or smaller sizes can be used depending upon the degree of accuracy desired. A dry 400 cc wide mouth jar with a screw cap is weighed and the weight (W₂) recorded to the nearest 0.001 gram. After drying, the tissue sample is placed immediately into the weighed 400 cc wide mouth jar and capped. Samples are allowed to cool to ambient temperature and the weight of the dry tissue sample and bottle (W₁) is determined to the nearest 0.001 gram. The bone dry weight of the tissue sample, (W_{d}), is then calculated from the equation (W₁−W₂). The jars with sample were then uncapped and placed in standard TAPPI conditions to equilibrate for 16 hours. After equilibration time is complete, the jars are capped and the weight of the conditioned tissue, jar and lid (W₃) recorded. In cases where air circulation into the container is an issue, it is preferred to remove the dried samples from the sample jar and allow the samples to equilibrate on a raised rack instead of within the container. After conditioning the sample is then returned to the jar, capped and weighed. The equilibrium moisture content (We) is then calculated from the equation (W₃−W₁). The percent equilibrium moisture is then calculated from the equation [(Wₑ/W_{d})*100]. The difference between the equilibrium moisture content of the plies containing the antimicrobial composition and plies not containing the antimicrobial composition is then determined by simple difference.

In the interests of brevity and conciseness, any ranges of values set forth in this specification contemplate all values within the range and are to be construed as written description support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5. In addition, any of the foregoing aspects of this invention can be further defined by any combination of one or more of the specified values and ranges recited for any properties described herein.

It will be appreciated that the foregoing description is given for purposes of illustration and that the scope of the invention is defined by the following claims and all equivalents thereto.

We claim:

1. A multi-ply tissue product comprising two outer plies and one or more inner plies containing an antimicrobial agent, wherein said one or more inner plies containing an antimicrobial agent have a geometric mean tensile strength which is less than the geometric mean tensile strength of the outer plies.

2. The product of claim 1 wherein the geometric mean tensile strength of one or more of the inner plies is from about 10 to about 75 percent less than the geometric mean tensile of the outer plies.

3. The tissue product of claim 1 wherein said one or more inner plies containing an antimicrobial agent also contain a deliquescent salt.

4. The tissue product of claim 1 wherein said one or more inner plies containing an antimicrobial agent also contain a debonder.

5. The tissue product of claim 1 wherein said one or more inner plies containing an antimicrobial agent are weakened by mechanical action.

6. The tissue product of claim 1 wherein said one or more inner plies containing an antimicrobial agent are weakened by selective calendering.

7. The tissue product of claim 1 wherein said one or more inner plies containing an antimicrobial agent are apertured.

8. The product of claim 1 wherein the antimicrobial agent comprises a carboxylic acid.

9. The product of claim 1 wherein the antimicrobial agent comprises a carboxylic acid and a surfactant.

10. The product of claim 7 wherein the antimicrobial agent comprises a carboxylic acid selected from the group of citric acid; malic acid; malic acid; tartaric acid; salicylic acid; glycolic acid; adipic acid; glutaric acid; succinic acid; benzoic acid; lactic acid; and mixtures thereof and the surfactant is an anionic surfactant selected from the group from the group of primary and secondary alkane sulfonates and sarcosinates.

11. The tissue product of claim 1 wherein said one or more inner plies containing an antimicrobial agent also contain a deliquescent salt, wherein the deliquescent salt is an inorganic salt selected from the group consisting of calcium chloride, lithium bromide, lithium chloride, sodium acetate, potassium acetate, ammonium acetate and mixtures thereof.

12. The tissue product of claim 1 wherein said one or more inner plies containing an antimicrobial agent comprise a deliquescent salt and an anionic surfactant, wherein the cationic species of the deliquescent salt and the cationic species of the anionic surfactant are of the same periodic table group.

13. The tissue product of claim 12 wherein the cationic groups are selected from the group IA metals.

14. The tissue product of claim 1 wherein said one or more inner plies containing an antimicrobial agent comprise a deliquescent salt and a cationic or non-ionic surfactant.

15. The product of claim 14 wherein the cationic species of the deliquescent salt is a group IIA metal.

16. The product of claim 14 wherein the anionic species of the deliquescent salt and the anionic species of the cationic surfactant are from the same periodic table group.

17. The tissue product of claim 14 wherein the cationic species of the deliquescent salt and the surfactant are the same.

18. The product of claim 1 wherein the moisture content of one or more inner plies containing the deliquescent salt is from about 30 to about 300 percent greater than the moisture content of the outer plies.

19. A method of making a multi-ply antimicrobial tissue product comprising:
a) providing two outer tissue plies and an inner tissue ply;
b) adding an antimicrobial agent to the inner ply;
c) chemically or mechanically weakening the inner ply; and
d) combining the two outer plies and the inner ply to form a multi-ply tissue product.

20. The method of claim 19 wherein the inner ply is chemically weakened by the addition of a deliquescent salt.

21. The method of claim 19 wherein the inner ply is mechanically weakened by calendering.

22. The method of claim 19 wherein the inner ply is mechanically weakened by aperturing.
